(19) United States
(12) Patent Application Publication
(10) Pub. No.: US 2012/0259154 A1
(43) Pub. Date: Oct. 11, 2012

(54) In Vivo Immunomagnetic Hyperthermia Platform for Any Cell or Virus Having a Target Surface Receptor

(75) Inventors: Bin Hong, West Lafayette, IN (US); Brian Markey, Park Forest, IL (US)

(73) Assignee: IVDiagnostics, Inc., Valperaso, IN (US)

(21) Appl. No.: 13/080,544

(22) Filed: Apr. 5, 2011

(51) Int. Cl. A61N 2/00; A61B 5/055; H01F 7/06; B32B 5/16; C07F 15/02; B82Y 5/00

(52) U.S. Cl. 600/12; 428/403; 544/225; 424/9.32; 335/209; 977/773

(57) ABSTRACT

The invention is a nano-entity conjugate for use in an in vivo immunomagnetic hyperthermia system for the detection and treatment of any cell or virus having a target surface receptor which encompasses a technology platform that can be used for both real-time monitoring of any cell or virus having a target surface receptor and as a delivery platform for certain types of treatment that are conducive for in vivo applications. The system allows of cell or virus enumeration; cell or virus capture; and cell or virus removal from the patient's circulatory system in-vivo using immunomagnetic hyperthermia. The application of immunomagnetic hyperthermia may actually diminish and eventually stop the progression of cancer and other blood borne or blood affected diseases.

### BACKGROUND OF THE INVENTION

1. Field of the Invention

The invention relates generally to a minimally invasive method and associated device for identifying, isolating and selectively destroying one or more selected cells or viruses having a targeted surface receptor, including, but not limited to, bacteria, viruses, cancer, normal animal cells, and altered or modified animal or bacteria cells. Among other fields and applications, the invention has utility in, the detection and eradication of circulating tumor cells (“CTCs”) in vivo.

2. Description of the Related Art

The effects of cancer related death is staggering. For example, each year in the United States, around one million new cases of cancer are diagnosed. Additionally, one out of every five people in this country will die from cancer or from complications associated with its treatment. Large amounts of money and time are directed to the research related to improving treatment and the better diagnosis of this disease.

It is known that most cancer patients are not killed by their primary tumor; instead patients are killed by the result of metastasis. Metastases are multiple widespread tumor colonies established by malignant cells that detach themselves from the original tumor and travel through the body. Quite often these metastases travel to distant sites. The detached tumor cells are CTCs.

Early detection of a primary tumor is ideal because it can often be eliminated by surgery, radiation, or chemotherapy or some combination of those treatments. However, the metastatic colonies are harder to detect and eliminate and it is often impossible to treat all of them successfully. Therefore, from a clinical point of view, metastasis can be considered the conclusive event in the natural progression of cancer. Moreover, the ability to metastasize is the property that uniquely characterizes a malignant tumor.

Based on the complexity of cancer and cancer metastasis and the frustration in treating cancer patients over the years, many attempts have been made to develop diagnostic tests to monitor metastasis.

For example, various systems are known in the art which assist physicians in determining whether to administer chemotherapy after tumor resection. Most of these systems include the assessment of the presence of microscopic metastatic disease by monitoring the presence of CTCs to determine cancer progression. These systems include: computed tomography, MRI, tissue/sentinel lymph node biopsy, serum cancer marker analysis, and flow cytometry, while other solutions include, size-exclusive filtration, density centrifugation, microfluidic chips, and immunomagnetic separation based technologies. Currently, the only in vitro technique approved by the FDA for human diagnostics of CTCs is Cell Search by Veridex (Raritan, N.J.). Cell Search is an immunocytochemical detection technique involving the extraction of small sample blood from a patient (7.5 ml) and subsequent removal of all leukocytes and extraneous cells from the blood sample to perform an analysis of just the CTCs in the sample.

This system involves the quantitation of the cells expressing epithelial markers after the isolation of the cells from about 5-20 ml of peripheral blood is extracted from the patient. Thus, this technique is an in vitro technique.

In addition to the requirement of extracting blood, there are other problems with the current in vitro detection techniques, which make it difficult to correlate the presence of CTCs to any disease progression. For example, there is a very small likelihood that the small sampling will have statistical significance. The number of the CTCs quantified from only 5-20 ml of extracted patient blood is very small when compared to the potential number of CTCs in a total volume of approximately 5 L blood in the average adult. This intrinsic defect may lead to poor or incorrect conclusions. For example, articles have documented that there is a low detection rate using the Cell Search system. Specifically, Stopeck et al. (Journal of Clinical Oncology, 2005); Riethdorf et al. (Clinical Cancer Research, 2007), Nole et al. (Annual Oncology, 2008); and Dawood et al. (Cancer 2008) each disclose studies in which patients with metastasis were tested using the CellSearch system. CTCs were only detected in 36-61% of the patients. Studies have also shown remarkably varying CTC counts, ranging from less than zero to thousands per ml in the same patient category. (Mostert, Cancer Treatment Reviews, 2009). It is presently unknown whether the difference in CTC counts is caused by cancer biology or varying sensitivity of the techniques used.

There are other unresolved issues with the current CTC detection methodologies that have caused caution when drawing conclusions from the data, such as, an unsatisfactory CTC detection rate, discordance across studies, and a focus reflecting tumor biology rather than tumor burden. For example, Roy et al. (Community Oncology, 2007) and Stopeck et al. (Journal of Clinical Oncology, 2005) conducted similar studies on 50 metastatic breast cancer (“MBC”) patients. Roy et al. detected CTCs in 54% of the patients, compared with 70% in the Cristofanilli et al. study. Thus, the utility of CTC in vitro testing to manage cancer is still unproven. In fact, The American Society of Clinical Oncology 2007 guidelines do not currently recommend CTCs as a risk predictor in the clinical practice.

The literature also discloses little to no correlation with the presence of CTCs and other clinical and/or pathological characteristics. For example, Lang et al. (Breast Cancer Research and Treatment, 2009) studied a group of 92 patients with operable breast cancers and neither tumor size, estrogen receptor status, progesterone receptor status, grade, histologic type, degree of nodal involvement, lymphovascular invasion, Ki-67, or marrow micrometastases displayed significant relationship with the presence of CTC. Only the HER2 status was correlated.

Despite the above-mentioned problems identified in the studies, the presence of five or more CTCs appears to indicate a significantly higher risk of progression at any stage of cancers. So, while the studies of CTCs show largely varied data, the use of CTCs as a diagnosis tool still shows promise.

In an effort to overcome some of the above-mentioned problems, He et al. disclosed an in vivo system for monitoring a patient through the use of a larger volume of the patient's blood. See He et al. “In vivo quantitation of rare circulating tumor cells by multiphoton intravital flow cytometry,” Proceedings of the National Academy of Sciences, 2007. He et al. specifically disclosed using a fluorescent label—folate conjugate which attaches to the folate receptor (FR) of CTC cells, and then using a probe to image the attached CTC cells. In developing the fluorescent label—folate conjugate, He et al. utilized the well known fact that cancer cells grow rapidly and require an increased supply of folate, a member of the vitamin B family, which plays an essential role in cell survival by participating in the biosynthesis of nucleic and amino acids. In fact, folate receptors are found up-regulated in 90% of ovarian and endometrial cancers, 86% of kidney cancers, 78% of non-small cell lung cancers, while folate receptors occur at very low levels in most normal tissues. The FR density also appears to increase as the stage of the cancer increases. Thus, to identify CTCs He et al. utilized a folate binding ligand. Then, the folate binding ligand was covalently conjugated to a fluorescent dye, and utilized in real-time in-vivo CTC imaging once it attached to the CTC. So, He et al. at least solved the problem of limited sampling. Using the real-time imaging techniques of He et al. more CTCs can be detected and enumerated.

So, while progress has been made which allows doctors to potentially probe significantly large volumes of blood samples from a cancer patient without requiring blood extraction, little progress has been made to develop a technique which also allows the in vivo treatment (i.e. destroying) of the CTCs. Given that metastasis is the leading cause for cancer death, responsible for 90% of all deaths in cancer patients, and CTCs are the driving force for carcinoma invasion and metastasis, the removal of CTCs could directly stop the intravasation and extravasation by CTCs.

The removal of other unwanted cell types and viruses in a patient would likewise be desired in the art. It is well known in the art that like CTCs, other cell types and viruses have surface receptors that may be used as targets for detection. Example of other unwanted cell types include, but are not limited to types of bacteria cells, erythrocytes, neutrophils, and leukocytes, including monocytes, lymphocytes, basophils, and eosinophils. Thus, the invention disclosed in lie et al. for detecting and imaging CTCs may be likewise be used for other cell types and viruses having a targeted surface receptor, including, but not limited to, bacteria, viruses, normal animal cells, altered or modified animal (such as CTCs) or bacteria cells, which we refer to herein collectively as “target xenocells”.

Thus, a method and associated device to destroy target xenocells is desired. It is known in the prior art that hyperthermia of cells through the use of magnetic particles is possible. The history of the use of hyperthermia with small particles in AC magnetic fields started in the late 1950s, but no clinical implication was above the horizon until more than three decades later. To utilize cell hyperthermia as a treatment tool, it was necessary to discover a vehicle to transport the magnetic particles to the cells. With the advancement of nanotechnology, also came particles small enough to inject into a patient. A colloidal dispersion of superparamagnetic iron oxide nanoparticles (SPION) exhibits an extraordinary absorption rate, which is much higher at clinically tolerable H_{0f} combinations in comparison to hysteresis heating of larger multidomain particles. So, with the knowledge that SPIONs have an extraordinary absorption rate and thus can absorb large amounts of heat, the present invention for the use of magnetic nanoparticles in a treatment to destroy cells evolved.

### SUMMARY OF THE INVENTION

An object of the invention is to provide a minimally invasive device that identifies, quantifies, isolates and selectively destroys one or more selected target xenocells.

Another object of the invention is to provide a system to identify target xenocells in vivo, monitor the target xenocells, and also destroy (intravitally cook) the target xenocells, through the use of target xenocells specific magnetic fluid hyperthermia (MFH) using target-specific probes chemically attached to magnetic nanoparticles.

Another object of this invention is to provide a multi-functional nano-entity conjugate comprising a target-specific probe, a magnetic nanoparticle and a fluorescent dye.

Another object of the invention is to provide a device comprising a high frequency alternating magnetic field (“AMF”) generator connected to a resonance circuit; wherein the high frequency generator comprises three induction coils containing electromagnets located at three poles equidistant from each other and a center; and a capacitor.

Another object the invention is to provide a device for detection and treatment of target xenocells, comprising a probe of fiber-optic array; a laser source for excitation of the conjugate; a signal detector; and an axial magnetic field generating device.

Another object of the invention is to provide system for detection and treatment of target xenocells comprising, injecting a nano-entity conjugate into the blood stream of a patient; applying AMF with an AMF generating device; collecting and isolating nano-entity conjugate immunomagnetically captured target xenocells; applying a rotating AMF with the AMF generating device; hyperthermically heating the immunomagnetically captured target xenocells; and intravitally cooking the immunomagnetically captured target xenocells.

Another object of the invention is to monitor the cell apoptosis during the hyperthermic heating by re-applying the AMF with the AMF generating device; placing a probe of fiber-optic array against a blood vessel on a skin surface of the patent; delivering a laser source from the fiber-optic array to excite the immunomagnetically attached to target xenocells; receiving fluorescence emission signals from the immunomagnetically captured target xenocells through the fiber-optic array; delivering the fluorescence emission signals to a detector, measuring the fluorescence emission signals through the detector; monitoring cell apoptosis in real-time via the detector; and de-activating the AMF generating device upon identification of apoptosis by morphological and other changes to the cells.

### BRIEF DESCRIPTION OF THE DRAWINGS

FIG. 1A of the drawings is an illustration of one synthetic pathway of the nano-entity conjugate in accordance with the present invention.

FIG. 1 of the drawings is an illustration of the nano-entity conjugate in accordance with the present invention.

FIG. 2 of the drawings is an illustration of the components of an axial magnetic field (AMF) generating device, which is a component of the device for the diagnosis and treatment of target xenocells in accordance with the present invention.

FIG. 3 of the drawings in an illustration of a preferred embodiment of the device for diagnosis and treatment of target xenocells in accordance with the present invention.

FIG. 4 of the drawings is an illustration of other components which may be used with the AMP generating device to comprise the device for diagnosis and treatment of the target xenocells in accordance with the present invention.

FIG. 5 of the drawings is an illustration of an alternative embodiment of the device for diagnosis and treatment of the target xenocells in accordance with the present invention.

FIG. 6 of the drawings is a schematic drawing reflecting the AMF generating device in accordance with the present invention.

FIG. 7 of the drawings is a drawing of a preferred embodiment of the cell structure imaging subsystem components located within the AMF generating device in accordance with the present invention.

FIG. 7A of the drawings is a schematic drawing of a preferred embodiment of the cell structure imaging subsystem components located within the AMF generating device in accordance with the present invention.

FIG. 8 of the drawings is an alternative embodiment of the cell structure imaging subsystem components in accordance with the present invention.

### DETAILED DESCRIPTION OF THE INVENTION

While the present disclosure may be embodied in many different forms, the drawings and discussion are presented with the understanding that the present disclosure is an exemplification of the principles of one or more inventions and is not intended to limit any one of the inventions to the embodiments illustrated.

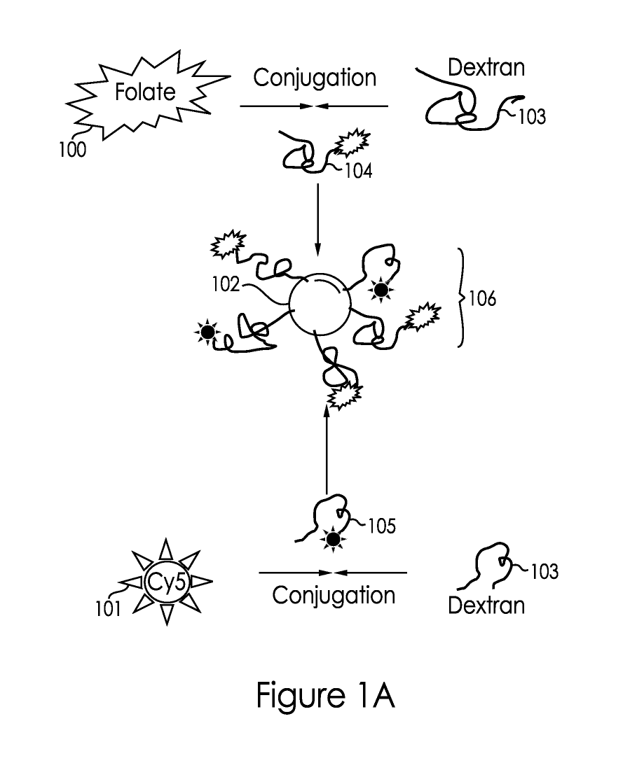

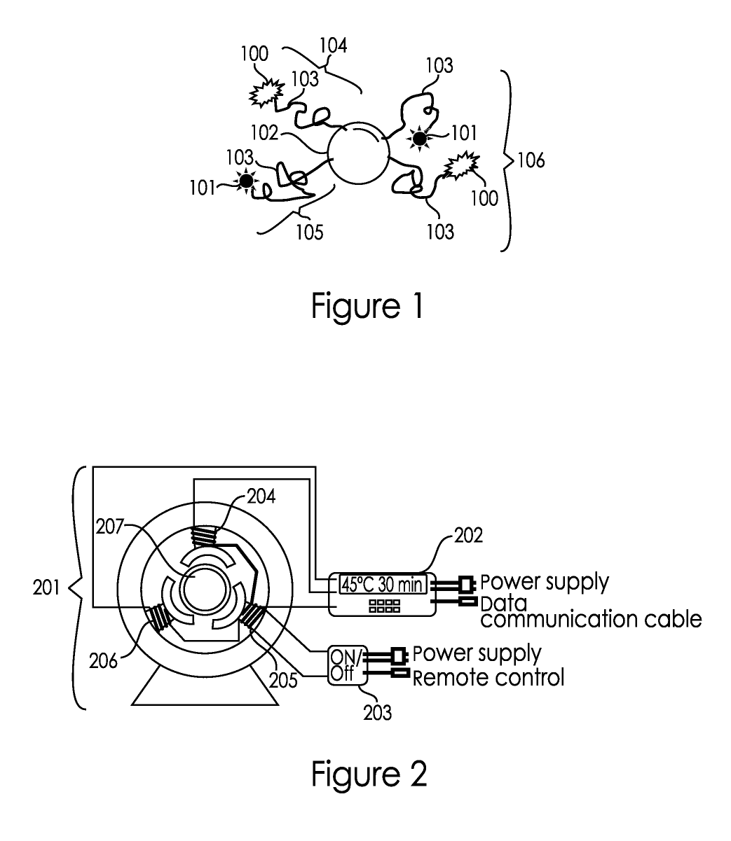

Prior to use of the system or device for diagnosis and treatment of target xenocells, the patient is given an intravenously injected chemically-bound nano-entity conjugate (106). As shown in FIG. 1, the nano-entity conjugate (106) should be comprised of at least target-specific probe (100), a fluorescent dye (101), and a magnetic nanoparticle (102). It is desired that the nano-entity conjugate (106) will comprise of target-specific probe (100) and fluorescent dye (101) chemically bound to a magnetic nanoparticle (102), or the nano-entity conjugate (106) will comprise of a target-specific probe (100)/fluorescent dye (101) conjugate chemically bound to a magnetic nanoparticle (102).

It is known in the art that target-specific probes can be created. For instance, probes have been created that take advantage of the observation that human carcinomas (one type of target xenocell) overexpresses a receptor for the vitamin folic acid. Generally, normal tissues either lack measurable folate receptors or express folate receptors at site which are inaccessible to parenterally administered drugs. Thus, radioactive or fluorescent folate conjugates have been injected in vivo to selectively label FR-expressing masses. Cancers which are presently known to express FR include, but are not limited to, ovarian cancers, endometrial cancers, some types of kidney cancers, and non-small cell lung cancers. Target-specific probes (100) are not limited to folate; rather any high-affinity, low-molecular-weight ligands with specificities for other rare populations of pathologic cells can be similarly designed and synthesized for use with a target-specific probe (100). These include, but are not limited to, bacteria, viruses, parasites, and activated immune cells.

It is desirable for the fluorescent dye (101) to be any fluorescent dye which may be injected into a living being preferably without causing damage at the concentration levels of dye contemplated for diagnosis and/or treatment. It is also desirable that the selected fluorescent dye chemically bind with either or both of the target-specific probe (100), and the magnetic nanoparticle (102). It is desired that the fluorescent dye (101) displays bright fluorescence at a relatively long emission wavelength. As an example, Cy5 is a red fluorescence dye favorable for cell imaging by minimizing the autofluorescence of cells in the wavelengths of its excitation and emission. Another example includes Oregon Green Dyes which are produced by Life Technologies Corporation (Carlsbad, Calif.).

If the nano-entity conjugate'(106) comprises a target-specific probe (100)/fluorescent dye (101) conjugate chemically bound to a magnetic nanoparticle (102), then it is preferred that the target-specific probe (100)/fluorescent dye (101) conjugate is covalently conjugated.

Generally, it is desired that the potential target-specific probe (100) and fluorescent dye (101) used for target xenocell labeling conjugate should exhibit at least the following properties: a high binding affinity and specificity towards the selected target xenocells; lack of immunity; fast blood clearance rate; and bright fluorescence at a relatively long emission wavelength. Specifically, a high binding affinity with specificity is desirable to selectively label the surface antigens of the selected target xenocells from among the millions of other blood cells. On the contrary, any non-specific binding above a background threshold may introduce false positive for the selected target xenocell. A lack of immunity is desirable because any labeling reagent that has a significant likelihood of triggering an immuno-response (particularly in a significant patient population) may compromise the detection sensitivity of the present method because this reaction would likely promote a rapid antibody-promoted clearance of the opsonized target xenocells by phagocytic cells (e.g. macrophage). It is desirable for the target-specific probe (100) and fluorescent dye (101) to have a fast blood clearance rate, so that it is cleared out of the circulation before any additional treatments. A longer blood clearance rate may cause significant interference from background fluorescence during subsequent treatments. Also, longer blood clearance time may comprise the detection sensitivity since the labeling reagents are uptaken by cancer cells and get quenched (e.g. endosome or lysosome degradation) if long washout time is needed. Lastly, bright fluorescence at a relatively long emission wavelength (e.g. near IR) helps to lower the tissue auto-fluorescence background.

For example, a folate-dye conjugate (or combination) is a good candidate because (1) folate-dye conjugates bind to folate receptor, a glycosylphosphatidylinositol-anchored glycoprotein, with subnanomolar affinity (K_{D}=10−₁₀M); (2) folate-dye conjugates have minimal immunity because they are small chemical molecules, (3) folate-dye conjugates are rapidly cleared from the blood (t_{1/2}=3.5 min for folate-FITC) following intravenous administration; and (4) if conjugated to appropriate fluorophore, folate-dye conjugates should exhibit desired optical properties (i.e. superior signal-to-background ratio). So, a nano-entity conjugate may include a folate-dye conjugate.

It is desirable for the magnetic nanoparticle (102) to be a good manufacturing practice (“GMP”) grade multi-functional superparamagnetic iron oxide nanoparticle (SPION). However, it is envisioned that the magnetic nanoparticle (102) can be any magnetic nanoparticle which can be chemically bound to either or both of an target-specific probe (100), or a fluorescent dye (101) and be optimized for use in the blood stream with an AMF generating device. It is important that the selected magnetic nanoparticle is not highly toxic or if it is undesirably toxic coated with a non-toxic protective coating with high mechanical strength. Ferromagnetic particles have been utilized in biologic systems in the past, but they have higher magnetic field strength, and thus a higher cost and may result in working difficulties.

The size (e.g. diameter and density) and composition of the magnetic nanoparticle (102) should be selected based on the amount of heat sought to be generated via the nanoparticle (102) interaction with the AMF. The efficiency of the heating by magnetic nanoparticles (102) in an AMF is defined by the specific loss power (SLP), which may vary by orders of magnitude in dependence on structural and magnetic particle properties of magnetic nanoparticles (102) on the one hand, and the amplitude and frequency generated by the AMF on the other hand. Though SLP (f,H) increases as a function of increased frequency f and field amplitude H in a wide parameter range, the enhancement of SLP (f,H) by increasing f and H is limited for technical, medical and economical reasons. For instance, for the first commercially developed AMP equipment for the treatment of human patients had its variable field amplitude limited to 18 kA/m at a fixed frequency of 100 kHz.

In the superparamagnetic size range of magnetic nanoparticles (102), SLP (f,H) is given by Equations (1) and (2),

SLP(f,H)=μ₀πχ″(f)H²f/ρ (1)

Where p is the mass density of the magnetic nanoparticle (102), f is the frequency, H is the field amplitude, μ₀ is the magnetic constant, and x(f) may be described by,

χ″(f)=χ₀φ/(1+φ²), φ=fτ_{R}, χ₀=μ₀M²ₛV/kT) (2)

Where Mₛ is the saturation magnetization, τ_{R} denotes the relaxation time, V represents mean particle volume, kT is the thermal energy.

Introducing here the condition f(H)=C/H (where C is the constant based on the patient type, target xenocell type, nanoparticle type, and other constituents) one gets the result that with increasing H (and correspondingly decreasing j) one approaches asymptotically the maximum SLP,

SLPₘₐₓ=μ₀πC²χ₀τ_{R}/ρ (3)

As a result, SLP increases in the superparamagnetic regime with increasing relaxation time τ_{R}, i.e. with increasing particle size, until the validity of the relaxation theory ceases near the transition to the stable single domain regime.

So, increasing SLP of magnetic nanoparticles (102) for MFH is important for successful implementation of the heat and to minimize dosage for use in clinical settings. In the prior art, Gonzales-Weimuller et al. found that substantially higher SLPs were achievable by decreasing the polydispersity and optimizing the size of the magnetic nanoparticles. At a constant frequency of 400 kHz with various ac-field amplitudes, highest SLP measured was 447 W/g at 24.5 kA/m for 11.2 nm particles.

Also, Chan et al. selected magnetic nanoparticles of approximately 15 nm in their study. Shinkai et al. have reported that particle size is a critical factor in obtaining a high SLP value. In Atsumi et al. study, maximum SLP was observed for the magnetic nanoparticles with an average particle diameter of 14 nm for an AMF of 3.2 kA/m at a frequency of 600 kHz.

Another study by Sharapova et al. demonstrated that the greatest value of the heat release at frequencies to 100 kHz (field 13.6 kA/m) was from the magnetic nanoparticles with an average size of approximately 16 nm.

So, while the desirable magnetic nanoparticle (102) size range is 11-16 nm based on the current commercially developed equipment for delivering AMF, the size of the magnetic nanoparticle (102) should be selected based on the equations herein to achieve the optimal heating efficiency for MFH, while also taking into consideration the desired amplitude and frequency of the AMF to achieve the desired results.

Besides modifying the size of the magnetic nanoparticle (102), the heating efficiency may be optimized by increasing the monodispersity of the magnetic nanoparticles (102), modifying the anisotropy of the magnetic nanoparticles (102) (shape or magnetocrystalline), and reducing the clustering of magnetic nanoparticles (102) due to strong magnetic interaction by modifying any magnetic nanoparticle coating. Besides the modifications listed herein, any modification to the magnetic particle (102) which optimizes the heating efficiency is contemplated.

It is also desired that the magnetic nanoparticle (102) selected has at least one of the following properties: a biodegradable and/or biocompatible surfactant coat, nontoxicity, biocompatibility, injectability, high-level accumulation in the target xenocells, and effective absorption of the AMF energy.

After selection of the target-specific probe (100), the fluorescent dye (101), and the magnetic nanoparticle (102) the individual components are chemically combined to create the nano-entity conjugate (106). FIG. 1 is an illustration of the nano-entity conjugate in accordance with the present invention. As is shown in FIG. 1, the nano-entity conjugate (106) is includes a tumor-specific probe (100) which is conjugated to a hydrophilic macromolecule (103), and results in a target-specific probe conjugate (104). The hydrophilic macromolecule (103) serves to (1) limit the magnetic core growth during the synthesis, (2) stabilize via sterical repulsions the magnetic nanoparticle dispersion in medium/blood and (3) reduce in vivo the opsonisation process. The nano-entity conjugate (106) also includes a fluorescent dye (101) conjugated to a hydrophilic macromolecule (103), which results in a fluorescent dye conjugate (105) The nano-entity conjugate (106) also includes a magnetic nanoparticle (102) to which both the target-specific probe conjugate (104) and the fluorescent dye conjugate (105) are chemically bound.

FIG. 1A is an example of the synthetic pathway of the nano-entity conjugate (106). In FIG. 1A, the target-specific probe (100) is folate. Specifically, the folate (100) will deliver the magnetic nanoparticles (102) to the target xenocells. The folate (100) also internalizes the magnetic nanoparticles (102) into the target xenocells via endocytosis for intracellular hyperthermia. In the target probe shown in FIG. 1A, the folate (100) is conjugated to a hydrophilic macromolecule (103). The hydrophilic macromolecule example provided in FIG. 1A is Dextran. The resultant product is target-specific probe conjugate (104). In the example of FIG. 1A, the fluorescent dye (101) is Cy5, a red fluorescence dye, which is favorable for cell imaging because it minimizes the autofluorescence of cells in the wavelengths of its excitation and emission. The fluorescent dye (101) is also conjugated to the hydrophilic macromolecule (103) Dextran. The resultant product is a fluorescent dye conjugate (105). Then, in the example of FIG. 1A, both the target-specific probe conjugate (104) and the fluorescent dye conjugate (105) are chemically bound to the magnetic nanoparticle (102) to create a nano-entity conjugate (106). It is contemplated that the individual components of the nano-entity conjugate (106) can be bound through several different chemical synthetic routes including, but not limited to, wet precipitation, co-precipitation, a reverse micelle mechanism, chemical vapor condensation, thermal decomposition and reduction, and liquid phase reduction.

After synthesis, it is preferred that the nano-entity conjugates (106) may be suspended in any physiologically acceptable buffer, and preferably a saline buffer with 0.1% sodium azide. It may be desirable to add an inert stabilizer to improve the shelf life of the nano-entity conjugates. Alternatively, the nano-entity conjugates (106) may be lyophilized and reconstituted.

The nano-entity conjugate (106) of FIG. 1 may be injected into a patient using a standard syringe directly into a vein or via a previously established IV port. It is envisioned that the patient may include, but is not limited to, a mouse, rat, horse, monkey, dog, cat, rabbit, or human. Following injection into the blood stream of the patient, the nano-entity conjugates (106) will attach to any target xenocells found in the blood stream via the target-specific probe, then the target xenocells will ingest the magnetic nanoparticles (102). By attachment, the target xenocells will also be fluorescently labeled with the fluorescent dye (101).

After injecting a patient with a nano-entity conjugate (106), a device for diagnosis and treatment of target xenocells such as the device illustrated in FIGS. 2-8 is capable of detecting, quantifying, isolating, monitoring, and removing target xenocells within a patient's circulatory system. It is contemplated that the device would be relatively portable, such that it can be moved to a location adjacent a patient's bed-side if so desired. It is contemplated that the device can be used as a Point-of-Care device capable of improving clinical outcome, while providing biometric data useful as a collaborative tool that can have significant complementary benefits when used with existing imaging, radiology and pathology protocols.

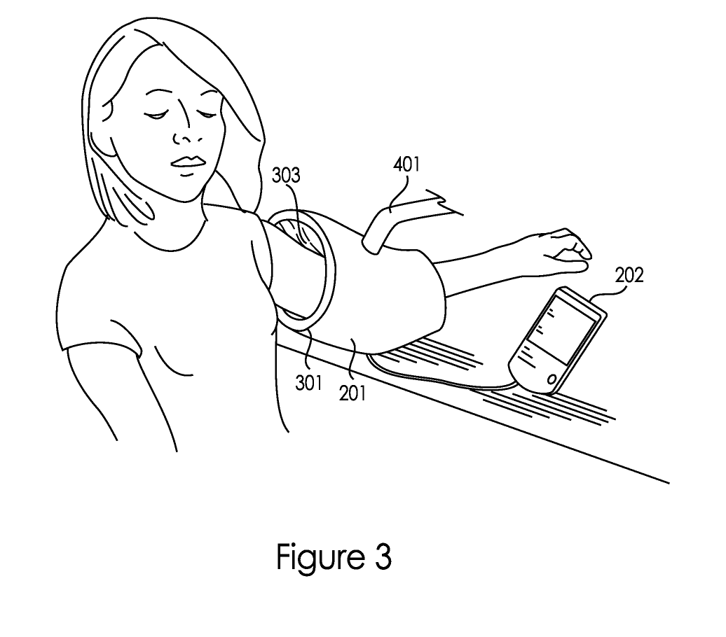

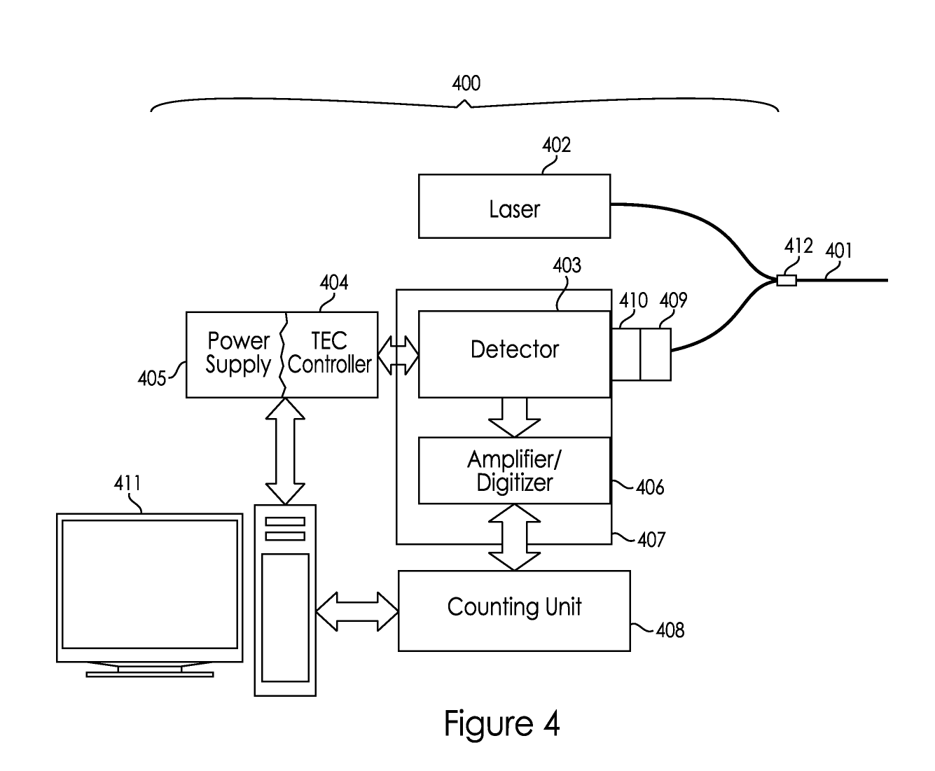

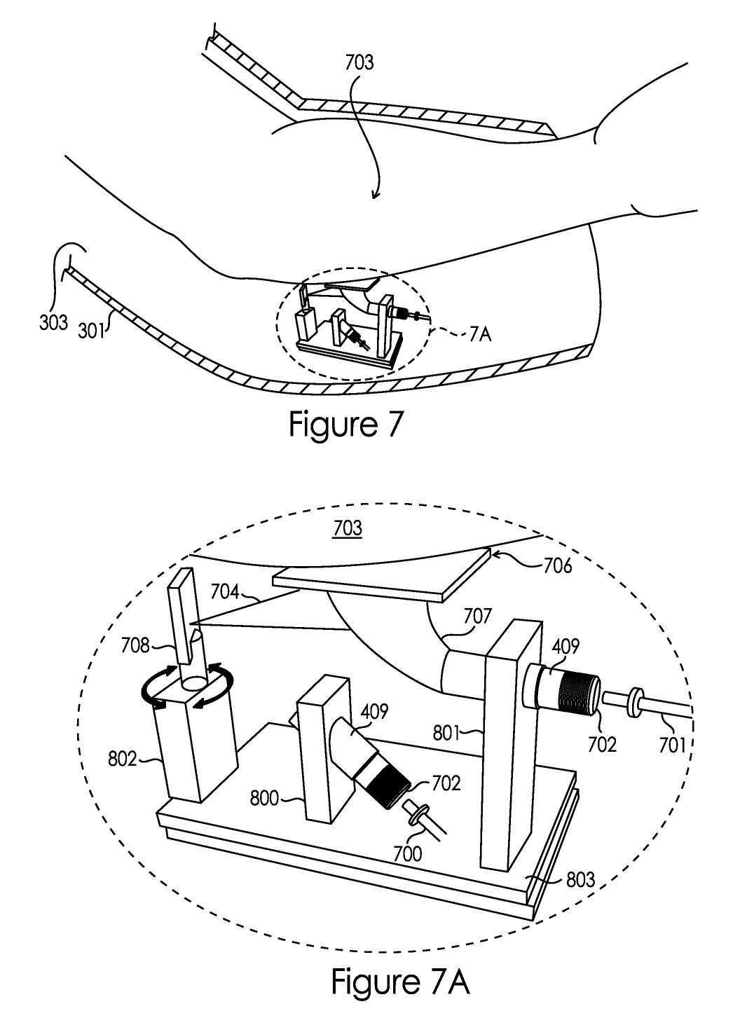

Generally, in a preferred embodiment, as shown in FIG. 2 and FIG. 3, the device, in part, consists of an AMF generating sub-system (201) which is contained within a sleeve (301), which may appear relatively similar to a blood pressure monitor cuff. In FIG. 3, the sleeve (301) encircles the patient's arm, but any extremity may be used. The device also has an imaging sub-system (400) as shown in FIG. 4, which may include at least a probe of fiber-optic array (401), a laser source (402), and a detector (403). As shown in FIG. 7, in a preferred embodiment, the imaging sub-system (400) is physically associated with and portions may even be contained within the sleeve (301) along with portions of the AMF generating sub-system (201).

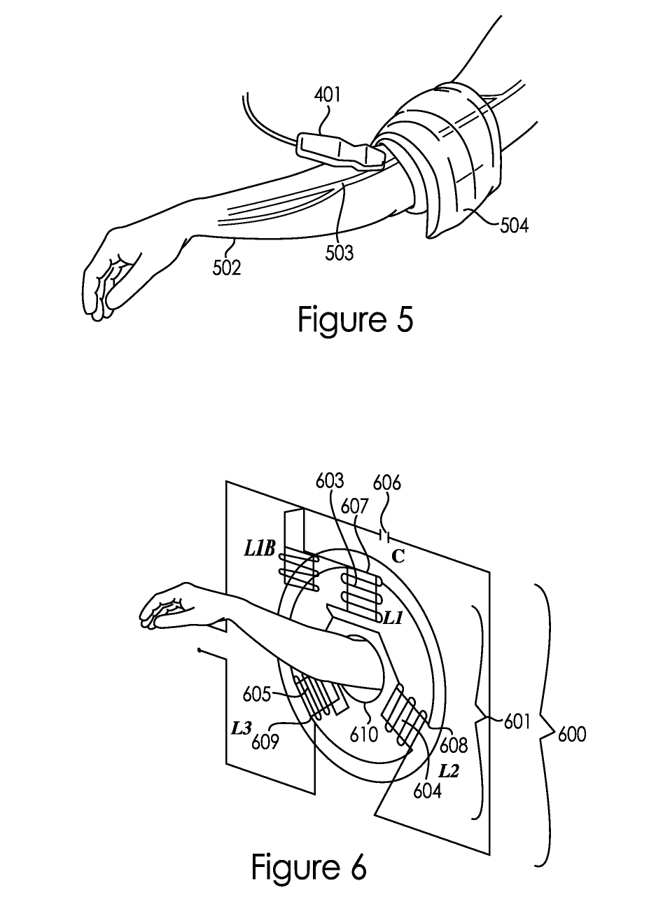

As shown in FIG. 3, in another embodiment the probe of fiber optic array (401) can be engaged through the gate of AMF generating device (201), and thus is removable. In an alternative embodiment the two systems can be mounted separately as shown in FIG. 5. In FIG. 5 another embodiment is shown wherein a fiber optic probe (501) containing the fiber optic array (401) is attached directly to the patient's arm (502). FIG. 5 reflects that the fiber optic array (401) is placed at the patient's arm (502) near a part of the circulatory system. Specifically, the fiber optic probe is attached on the patient's arm (502) in operable association with a blood vessel (503). The AMF generating device (201) is contained within a sleeve (504), which is similar to sleeve (301). It is contemplated that the selection of physical configuration will be determined based on the arrangement that provides better user-friendliness, safety of therapy, higher efficiency in control, as well as lower manufacturing cost.

FIG. 6 illustrates an AMF generating sub-system (600) for the detection and treatment of target xenocells. Preferably, the AMF generating device has two circuits: a static AMF circuit and a rotating AMF circuit. Generally, the AMF generating sub-system (600) comprises of a high frequency generator (601) operably connected to a static AMF circuit and a rotating AMF circuit, which are both resonance circuits. The resonance frequency (f) of the resonance circuits is preferably designed to generate frequencies in the range of approximately 100 kHz to 500 kHz. To activate the static AMF a voltage is applied from the high frequency generator (601) to induction coil L1 (603). The rotating AMF circuit has three poles (607, 608, 609) physically arranged at an angle of 120°, on which there are three induction coils with preferably ferrite cores containing electromagnets (603, 604, 605) that form a star connection. To activate the rotating AMF, the voltage is applied from the high frequency generator (601) to induction coil L1 (603), induction coil L2 (604) and induction coil L3 (605), through a capacitor (606), which ensures a phase shift of current equal to 120°. Thus, when the rotating AMF circuit is energized a sample containing the nano-entity conjugates (106) placed in the center (610) of the AMF generating device (600) is subject to the rotating AMF, which, in turn, create's heat. It is desired that the average internal temperature achieved in the bloodstream at the location of the nano-entity conjugates (106) is regulated using a device such as a thermocouple, laser, or some other suitable temperature (or average kinetic energy) measuring device. Alternatively, it is desired that the internal average temperature achieved in the bloodstream at the location of the nano-entity conjugates (106) be regulated by extrapolating that temperature from the average surface skin temperature.

FIGS. 2 and 3 also illustrate an AMF generating device (201). Particularly, FIG. 3 reflects a sleeve (301) through which a patient would place his arm or other extremity, such as a leg. Preferably, the AMF generating sub-system (201) is operably contained within the sleeve (301). The patient is or has previously been injected with a nano-entity conjugate (106). The nano-entity conjugate will be allowed to circulate in the patient for a period of time sufficient to allow the conjugate time to bind to a desired population of target xenocells. After this period of time, the AMF circuit is activated.

FIG. 2 illustrates the use of three induction coils (204, 205, 206) which are activated by a rotating AMF circuit to generate a rotating AMP substantially around the center (207) of the device, which is the location of the patient's extremity. FIG. 2 also depicts the operable connection of a separate power supply solely to a single induction coil. This separate power supply and the static AMF circuit activate induction coil L1 (204) of the AMP generating device. The resulting static AMF generates a relatively stable magnetic field of opposite polarity to the magnetic polarity of the nano-particles such that nano-particles disposed in sufficient proximity to the electro-magnetic field generated by L1 are held within the magnetic field such that the target xenocells attached to the nano-entity conjugates (106) are at least partially collected and functionally isolated within a section of the patient's bloodstream. It is contemplated that the static AMF will be selectively activated for a predetermined time dependent on many factors including by way of example, the age of patient, the medical history of the patient, and information regarding the target xenocell population. The predetermined time will essentially balance efficiency versus the building fluid pressure in the patient's veins that could overcome the magnetic forces attracting the nano-particles while further avoiding the substantial risk of a temporary, substantially total blockage of the patient's veins by the magnetically captured target xenocell population. It is contemplated that the collection time may be between 30-60 minutes depending on the answers to the foregoing variables (among others). After collection, the rotating AMF circuit will activate induction coil 1 (204), induction coil 2 (205) and induction coil 3 (206) to generate a rapidly rotating AMP, by which the isolated and collected magnetic nanoparticles (102) will become heat sources like thermoseeds, generating controllably released, target-specific safe heat via Brownian motion and/or Neel relaxation. It is also believed that the friction between the particles and the medium caused by the rotating AMF may result in a higher than theoretically anticipated heat release (due to a Brownian contribution). For a tumor cell, for example, the safe heat should be approximately 41-45° C. This target-specific safe heat causes the destruction of the target xenocells—held within the rotating AMF field—by cell apoptosis while surrounding cells are unaffected because the surrounding cells are not in physical contact with the nano-entity conjugate (106). After a predetermined length of time, which may be as long as 30 minutes, but potentially on the order of seconds, the rotating AMF circuit is deactivated and the static AMF circuit is reactivated to continue to hold the treated cells in place such that they may be analyzed for apoptosis via the imaging sub-system (400). Once apoptosis is successful, the dead target xenocells and their fragments are released into the circulation from their confinement in the AMF. Then, the apoptosis target xenocells would be naturally eliminated by the patient's body via phagocytosis. The magnetic nanoparticles uptaken by the target xenocells are degraded in the macrophage lysosomal compartment within days and eventually incorporated into the body's iron repository.

It is contemplated that the AMF frequency has to be higher than 50 kHz to minimize neuromuscular electrostimulation but lower than 10 MHz for appropriate penetration depth of the RF-field within the patient's tissue.

As shown in FIG. 4, in addition to the AMF generating device (201) there are several components to the device for diagnosis and treatment of target xenocells. Specifically, the device's imaging sub-system (400), may include a probe of fiber-optic array (401), a laser source (402), and a detector (403). The imaging system (400) allows for enumeration/quantification of the target xenocells and monitoring of the cell apoptosis progress during (and after) the AMF hyperthermia process. As indicated above and shown in FIG. 3, the fiber-optic array (401) of the imaging sub-system (400) may be inserted within the same housing as the AMF generating device (201), for example the sleeve (301), or as shown in FIG. 5 the fiber-optic array (401) of the imaging system (400) may be disposed physically separate from the AMF generating device (201). However, even if separate, the fiber-optic array (401) must be disposed such that it visualizes the cells held within the magnetic field created by the AMF generating device (201).

It is contemplated that portions of the imaging sub-system components may be housed together in an enclosure. For example, as shown in FIG. 4 it is possible for the detector (403) and the amplifier (406) to be housed in a module (407), but separate from the laser (402). It is also anticipated that computer (411) will be housed separately, but it is not required.

A laser source (402) is provided for excitation of nano-entity conjugates (106) that label the target xenocells. Specifically, the laser excites the fluorescent dye (101), which are attached to the target xenocells. It is contemplated to use either multiphoton or confocal fluorescence imaging to examine the fluorescent target xenocells. The use of continuous-wave lasers at 488 nm, 543 nm, and 780 nm is preferred, and allows for time-lapsed measurements. A ultrafast laser for multiphoton excitation may also be used. It is also contemplated that an inverted laser scanning microscope coupled with a Tl:Sapphire laser may be used.

A probe of fiber-optic array (401) is provide for laser delivery. By using a probe of fiber-optic array (401), the cells in the blood vessel can be scanned at rates 500-1000 times higher than the conventional digital microscope, due to a large field of view (50 mm). This enables a real-time analysis of cell images, as well as target xenocell enumeration. It is desired that the probe of fiber-optic array (401) is furcated. As an example the probe of fiber-optic array (401) can be double-clad photonic crystal fiber (PCF).

An example of a preferable PCF includes, but is not limited to, DC-165-16-passive by Crystal Fibre Inc. The inner core of the DC-165-16-passive fiber has a diameter of 16 μm which ensures good coupling of the laser beam. The inner cladding of the DC-165-16-passive fiber has a diameter of 165 μm with a high numerical aperture of 0.6 for efficient signal collection. Alternatively, an equivalent PCF can be utilized, which may have similar characteristics.

After laser delivery, the same probe of fiber-optic array (401) may be used for signal collection. The probe of fiber-optic array (401) detects the fluorescence signal from the fluorescently labeled target xenocells. It is contemplated that the detected fluorescence signal may be separated from the laser by a dichroic mirror (412), collected by the probe of fiber-optic array (401), and detected by a photon detector (403). The purpose of this design is to produce the smallest fiber head, which is critical for intravascular applications.

It is contemplated that the probe of fiber-optic array (401) can be put against a blood vessel on the skin surface to deliver the laser source (402) to the site of target and receive fluorescence emission signals to the detector (403).

It is contemplated that the imaging system may include a collimating lens (409) which focuses the diverging light from the probe of fiber-optic array (401) to parallel light rays in order to achieve a higher degree of optical coupling to an optical filter (410) and the detector (403).

It is also contemplated that the imaging system may include an optical filter (410) which is used to selectively measure specific wavelengths of light and reject those wavelengths not desired for measuring. An example of a preferred optical filter (410) includes, but is not limited to, Chroma HQ675/50 which is an optical filter with a center wavelength of 675 nm and a bandwidth of 50 nm. This filter passes light between 650-700 nm and rejects light outside that range.

A high-sensitivity photon detector is preferred for detection and quantification of the target xenocells. The contemplated detector (403) is an avalanche photodiode (APD) or photomultiplier tube (PMT) and is used to measure the fluorescence signals.

It is contemplated that the PMT is electrically cooled using a thermoelectric cooler (TEC) (404) and a power supply (405) and has a quantum efficiency of 0.4 at 600 nm. The PMT should detect a fluorescence signal at around 600 nm. An example of a preferable PMT includes, but is not limited to, Hamamatsu PMT (cat #H7422-40) in conjunction with a C9744 photon counting unit to convert PMT output to CMOS 5v output pulses for counting. Alternatively, an equivalent PMT can be utilized, which may have similar characteristics. For detection of a fluorescence signal beyond 700 nm, an example of a preferred PMT to employ is a PMT provided by Hamamatsu (cat #R3896), which has a quantum efficiency of 0.15 until 900 nm. The PMT can be modified accordingly to improve the signal-to-background ratio. Generally, the PMT measures the fluorescence.

As an alternative to using a PMT, a high efficiency APD could be used. It is contemplated that the APD would incorporate a TEC (404) and would have a peak detection efficiency of 65% at 650 nm. The APD should detect fluorescence signals in the range of 600 nm and above. An example of a preferred APD, includes, but is not limited to, the SPCM-AQR series from Perkin Elmer. The module provides TTL output pulses corresponding to photon detection events.

The imaging subsystem (400) has at least two subsystems, one for enumerating the target xenocells, and one for imaging the cellular structure of the target xenocells. As shown in FIG. 4, the enumeration subsystem (i.e. counting system) of the imaging subsystem (400) includes at least a fiber-optic array (401), a laser source (402), a detector (403), an amplifier (406), a counting unit (405), and a computer (411). The counting subsystem identifies and counts the target xenocells as they travel through the bloodstream and past the fiber-optic array (401). It is contemplated that the counting subsystem is activated prior to activating the AMF generating device (201) to quantify the target xenocells present in the bloodstream.

For example, as shown in FIG. 4, it is envisioned that once the fluorescence has been measured by the detector (403), the signal may be amplified and converted to digitized, 5v pulses by an amplifier (406). An example of a preferred amplifier (406) includes, but is not limited to, the Hamamatsu C9744 which has an output linearity up to 10̂7 pulses/second. It is desired that the detector (403) and the amplifier (406) can be housed in a self contained module (407). Then, the pulses are counted by a high speed, gated counting unit (405) interfaced to the computer (411). An example of the counting unit (408) includes, but is not limited to, a Hamamatsu C8855-01 which is a zero dead time, double counter capable of counting signals up to 50 MHz with a selectable gate width from 50 us to 10 s. Collected signal counts are sent to a computer (411) for analysis. Target xenocells quantification and a fluorescence intensity trace is produced. The target xenocells will display as spikes in the trace.

It is contemplated that the signal and background fluorescence intensities and the signal-to-background ratios are quantified on single cells by using software as the cells are detected. Also, the blood vessel fluorescence intensities are quantified in each image by calculating the mean fluorescence using the same software. An example of an appropriate software program includes, but is not limited to, FlowView Software by Olympus, version 4.3. Additionally, quantitation of the target xenocells occurs by collecting of digitized signals during scanning, exporting the signals, and analyzing the signals by a software program. An example of an software program with these capabilities includes, but is not limited to, MATLAB 7.0 platform. The software program used for analysis should also be able to eliminate high-frequency noise with a fast Gaussian filter, and visually present the fluorescent cells.

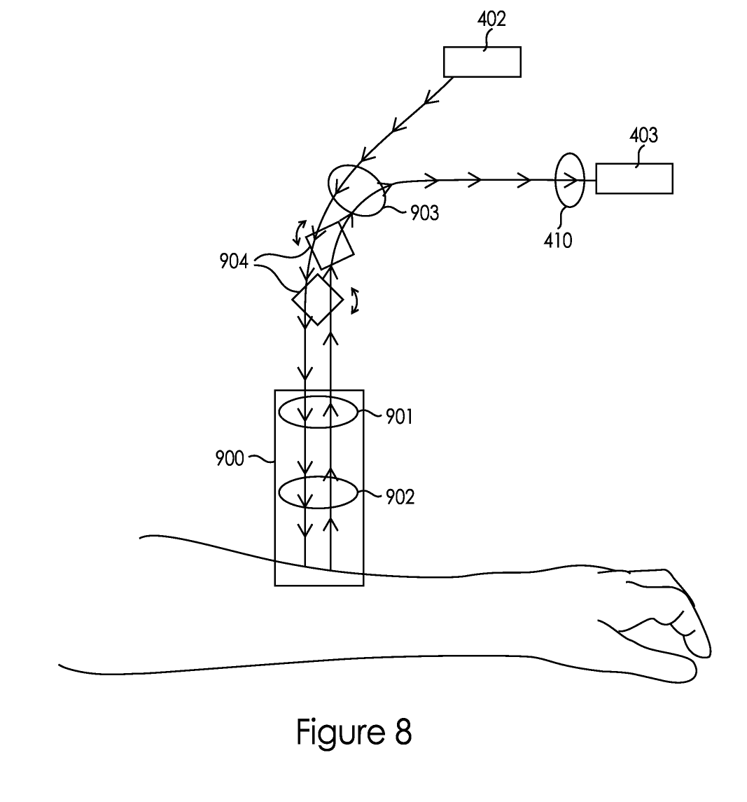

The other subsystem of the imaging subsystem (400) is the cell structure imaging subsystem as partially shown in FIG. 7 and FIG. 8, which includes various components, including at least a fiber-optic array (401), a laser source (402), a detector (403), and a computer (411). The cell structure imaging subsystem may be activated after the static AMF circuit is activated to image the cells which have accumulated in the AMP. Once the cells are imaged, the cell imaging subsystem may be turned off. The cell imaging subsystem may also be activated after the rotating AMF circuit is activated and deactivated. This is done to obtain a post-hyperthermia image of the target xenocalls, and analyze any morphological changes to the cells.

As shown in FIG. 3, the fiber-optic array (401) is coupled to the AMF generating device (201). Specifically, as shown in FIG. 7 and FIG. 8, the fiber-optic array (401) is coupled to the AMF generating device (201) via the laser source branch (700) and the detector branch (701). The laser source (402) and the detector (403) are not shown in FIG. 7 and FIG. 8 as they are located remotely from the patient, outside the AMF generating device (201), either in one enclosure containing both, or separately.

FIG. 7 and FIG. 7A specifically show the part of the cell structure imaging subsystem which is located within the AMF generating device (201), which comprises of at least the movable plate (803), the fiber connectors (702), the rotating mirror (708), and the clear window (706) through which a patient's skin (703) is scanned. In one embodiment, the components identified in FIG. 7A are located between the outer layer of the medical sleeve (301) and the AMF generating device chamber (303) at any location within the AMF generating device (201). It is envisioned that each AMF generating device (201) would have one set of the components identified in FIG. 7A, and thus, while it is possible to image the top, bottom, or side of arm dependent on the location of the actual components, each AMF generating device (201) would have its own specific design and only be able to image one surface of the arm. Likewise, as shown in FIG. 3, dependent on the actual location in the sleeve (301) of the components identified in FIG. 7A, the fiber-optic array (401) can be inserted at any convenient location into sleeve (301) for easy connection of the laser source branch (700) and the detector branch (701) of the fiber-optic array (401) to the fiber connectors (702).

As shown in FIG. 7A, the laser source branch (700) and the detector branch (701) of the fiber-optic array (401) are connected to the AMP generating device (201) via fiber connectors (702). A threaded type fiber optic connector (SMA) is preferred. As shown in FIG. 8, the fiber connectors (702) are mounted on the laser small lens holder (800) and the detector small lens holder (801) within the AMF generating device (201). A rotating mirror (708) is also mounted on the rotating mirror small lens holder (802). It is contemplated that the laser small lens holder (800), the detector small lens holder (801), and the rotating mirror small lens holder (802) are each affixed to a movable plate (803). As shown in FIG. 7A, the laser source (402) via the laser source branch (700) directs a laser beam (704) from the laser source (402) to a rotating mirror (708) which causes the laser beam (704) to scan the patient's extremity, specifically the patient's skin surface (703) in a horizontal side to side direction through a clear window (706).

Each horizontal scan of the laser beam (704) produces a single line of pixels which is detected by the detector (403) through the light collector fiber optic bundle (707) of the detector branch (701) which is positioned directly below the clear window (706). The single line of pixels is used to create the final image. To take multiple scans through the clear window (706) the movable plate (803) moves in a vertical motion which moves the laser beam (704) already moving in a horizontal side to side motion along the clear window (706). It is contemplated that the movable plate (803) is stepper motor driven stage. As shown in FIGS. 7, 7A, a collimating lens (409) may be coupled to the fiber connectors (702) to focus light onto the detection branch (701). The vertical movement of the movable plate (803) and the horizontal movement of the laser beam (704) creates a complete image of the target xenocells within a blood vessel of the patient's extremity, which allows the user to analyze the morphology of the target xenocells by using imaging computer software.

In another embodiment as shown in FIG. 8, a confocal microscope may be utilized in the cell structure imaging subsystem within the AMF generating device (201). The laser source (402) and the detector (403) are located remotely from the patient, separate from the AMF generating device chamber (303), and either in one enclosure, or separate from each other. The cell imaging subsystem components which scan the patient's skin (703), and are integrated into the AMF generating device (201) and comprise of at least a dichroic mirror (903), an optical filter (410), scanning mirrors (904), and an inverted microscope (900). The inverted microscope has an eyepiece (901) and an objective (902). A rasterized image of the target xenocells is formed as the scanning mirrors (904) scan the beam from the laser source (402) horizontally and vertically over the patient's extremity, an image is directed back to the scanning mirrors (904) and the dichroic mirror (903) to the detector (403), and the detector (403) output intensity is correlated to the laser source (402) beam position. The fiber-optic array is not furcated in this embodiment.

Also, in a preferred embodiment, as shown in FIGS. 2 and 3, an I/O module (202, 203) may be added to the imaging subsystem (400). For example, an LED displays can be incorporated for viewing the skin temperature and the elapsed heating time. As shown in FIG. 2, control panels 1 (202) and 2 (203) can provide manual control of the axial magnetic field and rotating magnetic field; respectively. It is contemplated that the control to the magnetic fields can also be programmed and remotely activated/deactivated through data communication cable or wirelessly.

It is contemplated that through the combination of the nano-entity conjugate and the device for the diagnosis and treatment of target xenocells, a system for the diagnosis and treatment of target xenocells can be developed. Specifically, as shown in FIG. 3, during the treatment, the patient inserts an extremity (302) to the chamber (303) of the AMF generating device (201). Generally, first the patient is administered the nano-entity conjugate (106). The nano-entity conjugates (106) fluorescently label the target xenocells with the fluorescent dye (101) when the target-specific probe (100) attaches to a receptor on the target xenocell. Then attachment causes the magnetic nanoparticle (102) to be internalized into the target xenocells via endocytosis. Upon activation of the static AMF circuit of the AMF generating device (201) the target xenocells are collected in the blood vessels via AMF, located in the chamber (303), under the AMF generating device (201). Next, the rotating AMF circuit of the AMF generating device (201) is activated to create a rotating AMF for heating therapy. Basically, the AMF isolates and enriches the target xenocells by the magnetic field, and destroys them intravitally thereafter via AMF generated heat. Lastly, after a predetermined period of time, the static AMF circuit of the AMF generating device (201) is activated again to hold target xenocells in place for analysis and monitoring of the hyperthermia process. At that point, the imaging system (400), comprising at least that probe of fiber optic array (401), laser source (402), and a detector (403) are used to confirm the death of the target xenocells in terms of their cell morphology and integrity in real-time. During apoptosis that target xenocells fragment, thus, target xenocells destroyed by the intravitally hyperthermia with show morphological changes. The heating therapy is then stopped by deactivating the AMF. The dead target xenocells and their fragments are released to the circulation from the confinement of the AMF. Then, the target xenocells are eliminated via phagocytosis. The magnetic nanoparticles (102) uptaken by the target xenocells are degraded in the macrophage lysosomal compartment within days and eventually incorporated into the body's iron repository.

It is envisioned that the device and/or system for the diagnosis and treatment of target xenocells may be expanded to several other applications.

For example, the system for the diagnosis and treatment of target xenocells may be expanded as a way of isolating the CTCs, and selectively extracting and analyzing the CTCs for genetic mutations, thus assisting oncologists and pathologists with the analysis of individual tumor cells. Particularly, the system may be used for CTC capture to assist in the capture of rare tumor cells for CTC diagnosis.

Likewise, the system for the diagnosis and treatment of target xenocells may be expanded as a way of isolating any cell or virus, and selectively extracting and analyzing the cell or virus for genetic mutations, thus assisting doctors with the analysis of individual cells or virus types. Particularly, the system may be used to assist in the capture of rare cell and virus types for diagnosis.

The device for the diagnosis and treatment of target xenocells may be expanded for use with other specific tagging of tumor types and cell types to address the diagnosis, isolation and removal of other blood borne diseases, and infectious diseases and viruses.

The device may be used for enumeration/monitoring to complement imaging, biopsies, and specialized scans.

The device may be expanded for immunomagnetic therapy on target xenocells.

The device may be expanded to couple with chemotherapy, or any other type of therapy to treat target xenocells.

The device may be expanded to identify, quantify, isolate and selectively destroy and/or treat cells relating to or causing inflammatory diseases, infectious diseases, arthritis, colitis, irritable bowel syndrome, bacterial infections, and tropical diseases.

The foregoing description and drawings merely explain and illustrate the invention and the invention is not limited thereto. While, the specification in this invention is described in relation to certain implementation or embodiments, many details are set forth for the purpose of illustration. Thus, the foregoing merely illustrates the principles of the invention. For example, the invention may have other specific forms without departing from its spirit or essential characteristic. The described arrangements are illustrative and not restrictive. To those skilled in the art, the invention is susceptible to additional implementations or embodiments and certain of these details described in this application may be varied considerably without departing from the basic principles of the invention. It will thus be appreciated that those skilled in the art will be able to devise various arrangements which, although not explicitly described or shown herein, embody the principles of the invention and, thus, within its scope and spirit.

What is claimed is:

1. A multi-functional chemically bound nano-entity conjugate comprising of at least:
a target-specific probe;
a magnetic nanoparticle; and
a fluorescent dye.

2. A multi-functional chemically bound nano-entity conjugate of claim 1, wherein the target-specific probe is folate.

3. A multi-functional chemically bound nano-entity conjugate of claim 1, wherein the target-specific probe is covalently conjugated to the fluorescent dye before it is chemically bound to the magnetic nanoparticle.

4. A multi-functional chemically bound nano-entity conjugate of claim 1, wherein the magnetic nanoparticle has at least one of the following properties: a biodegradable surfactant coat, a biocompatible surfacant coat, nontoxicity, biocompatibility, injectability, high-level accumulation in the target cells or viruses having a target surface receptor (“target xenocells”), or an effective absorption of the energy of axial magnetic field (AMF).

5. A multi-functional chemically bound nano-entity conjugate of claim 1, wherein the magnetic nanoparticle is a superparamagnetic iron oxide nanoparticle (SPION).

6. A multi-functional chemically bound nano-entity conjugate of claim 1, wherein the size magnetic nanoparticle is directly correlated to the amount of heat required by the AMF.

7. A multi-functional chemically bound nano-entity conjugate of claim 1, wherein the fluorescent dye is Cy5.

8. A multi-functional chemically bound nano-entity conjugate of claim 1, wherein at least one of the target-specific probe or the fluorescent dye is conjugated with a hydrophilic macromolecule.

9. A multi-functional chemically bound nano-entity conjugate of claim 1, wherein the nano-entity conjugate is suspended in at least a saline buffer with a physiologically acceptable buffer.

10. An axial magnetic field (AMF) generating device comprising:
a high frequency generator connected to a resonance circuit;
wherein the high frequency generator comprises three induction coils containing electromagnets located at three poles equidistant from each other and a center; and
a capacitor.

11. An AMF generating device of claim 10, wherein the device has at least two circuits.

12. An AMF generating device of claim 11, wherein one circuit is the static AMF circuit.

13. An AMF generating device of claim 12, wherein the static AMF circuit is activated activating one induction coil.

14. An AMP generating device of claim 13, wherein the activation of one induction coil generates a static AMF.

15. An AMF generating device of claim 14, wherein the generated static AMP magnetically collects and isolates magnetic nanoparticles.

16. An AMF generating device of claim 11, wherein one circuit is the rotating AMF circuit.

17. An AMF generating device of claim 16, wherein when the rotating AMP circuit is activated voltage is applied to the three induction coils.

18. An AMF generating device of claim 17, wherein the application of voltage to the three induction coils generates rotating AMF.

19. An AMF generating device of claim 18, wherein the rotating AMF creates heat.

20. A device for diagnosis and treatment of cells or viruses having target xenocells comprising:
an AMP generating device;
a probe of fiber-optic array;
a laser source for excitation of a nano-entity conjugate; and
a signal detector.

21. The device according to claim 20, which further comprises a multi-function I/O module.

22. The device according to claim 21, wherein the multi-function I/O module includes at least one of the following; a temperature display or a controller for the AMF.

23. The device according to claim 20 wherein the probe of fiber-optic array is a double-clad photonic crystal fiber (PCF).

24. The device according to claim 23 wherein the double-clad PCF is equivalent to DC-165-16-passive, Crystal Fibre Inc.

25. The device according to claim 20 wherein the probe of fiber-optic array is capable of delivering a light source and receiving signal.

26. The device according to claim 25, wherein the signal is a fluorescence emission.

27. The device according to claim 26 wherein the signal is separated from the laser source by a separation means.

28. The device according to claim 20 wherein the laser source provides continuous-wave layers at 488 nm, 543 nm, and 780 nm.

29. The device according to claim 20 wherein, the signal detector is a high-sensitivity photon detector.

30. The device according to claim 29 wherein, the high-sensitivity photon detector is an electrically cooled photomultiplier tube (PMT).

31. The device according to claim 30 wherein, the PMT is electrically cooled using a thermoelectric cooler (TEC).

32. The device according to claim 31 wherein the electrically cooled PMT has a quantum efficiency of 0.4 at 600 nm.

33. A device according to claim 31 wherein the PMT will detect a fluorescence signal at around 600 nm.

34. The device according to claim 20 wherein the signal detector is an avalanche photodiode (APD).

35. The device according to claim 34 wherein the APD incorporates a TEC.

36. The device of claim 20 further comprising at least one or more of an collimator, an optical filter, an amplifier, a counting unit, and a computer.

37. The device of claim 36, wherein the amplifier is housed in a module with the detector.

38. The device according to claim 20 wherein upon signal detection the signal is sent to a computer and a fluorescence intensity trace is produced.

39. The device according to claim 20 wherein the probe of fiber-optic array, the laser source for excitation of a nano-entity conjugate, and the signal detector count the target xenocells in a patient's bloodstream.

40. The device according to claim 20 wherein the probe of fiber-optic array, the laser source for excitation of a nano-entity conjugate, and the signal detector image the cell structure of the target xenocells.

41. A system for diagnosis and treatment of target xenocells comprising:
injecting a nano-entity conjugate into the blood stream of a patient;
applying AMP with an AMF generating device;
collecting and isolating nano-entity conjugate immunomagnetically captured target xenocells;
applying a rotating AMF with the AMF generating device;
hyperthermically heating the immunomagnetically captured target xenocells; and
intravitally cooking the immunomagnetically captured target xenocells.

42. The system for diagnosis and treatment of target xenocells of claim 41 further comprising:
re-applying the AMF with the AMF generating device;
placing a probe of fiber-optic array against a blood vessel on a skin surface of the patent;
delivering a laser source from the fiber-optic array to excite the immunomagnetically attached to target xenocells;
receiving fluorescence emission signals from the immunomagnetically captured target xenocells through the fiber-optic array;
delivering the fluorescence emission signals to a detector,
measuring the fluorescence emission signals through the detector; and
monitoring cell apoptosis in real-time via the detector.

43. The system according to claim 41 wherein the patient is an animal.
